(19) United States
(12) Patent Application Publication
(10) Pub. No.: US 2008/0221023 A1
(43) Pub. Date: Sep. 11, 2008

(54) Use of Protein Hydrolysates For the Manufacture of a Medicament For Prophylaxis and/or Treatment of a Dpp-IV Mediated Condition

(75) Inventor: Jan-Willem Pieter Boots, Bilthoven (NL)

Correspondence Address: THE WEBB LAW FIRM, P.C., 700 KOPPERS BUILDING, 436 SEVENTH AVENUE, PITTSBURGH, PA, 15219, US

(73) Assignee: Campina Nederland Holding, Zaltbommel (NL)

(21) Appl. No.: 11/597,722

(22) PCT Filed: May 24, 2005
(86) PCT No.: PCT/EP2005/005757

§ 371 Date: Jan. 2, 2008

(30) Foreign Application Priority Data
May 27, 2004 (EP) 04076579.4

(51) Int. Cl. A61K 38/16; A61P 3/00; A61P 19/02

(52) U.S. Cl. 514/12

(57) ABSTRACT

Disclosed is the use of one or more protein hydrolysates for the manufacture of a medicament, food supplement, beverage or food product for prophylaxis and/or treatment of a DPP-IV mediated condition, like, for example, obesity, type II diabetes mellitus, autoimmune diseases.

The present invention relates to the use of one or more protein hydrolysates for the manufacture of a medicament, food supplement, beverage or food product for prophylaxis and/or treatment of a DPP-IV mediated condition.

Dipeptidyl peptidase IV (DPP-IV) is a multifunctional transmembrane glycoprotein that contains N-terminal serine dipeptidase activity. It is present on most mammalian cells, in a variety of tissues such as liver, kidney, small intestine, salivary gland, blood cells and plasma. Little is however known about the physiological role of DPP-IV.

DPP-IV has been implicated in cellular processes involving immune, inflammatory and endocrine functions. In vitro, DPP-IV has been shown to cleave many hormones and chemokines, such as e.g. glucagon-like peptide 1 (GLP-1).

GLP-1 is an incretin hormone that is released postprandially. GLP-1 has multifaceted actions, including glucose-induced stimulation of insulin biosynthesis and secretion, inhibition of glucagon secretion, regulation of gene expression, trophic effects on β cells, inhibition of food intake, and slowing of gastric emptying. These effects contribute to the normalisation of elevated blood glucose, as well as to the control of satiety and body weight. GLP-1 has been shown to reduce postprandial and fasting glycemia in subjects with type 2 diabetes mellitus and may therefore be a potentially useful new therapeutic agent in the treatment of type 2 diabetes mellitus. Moreover, GLP-1 could be used to increase satiety and also to prevent en treat obesity. See e.g. Conarello, S. L. et al. 2003. Proc. Nat. Acad. Sci. USA, vol. 100:6825-6830; Deacon, C. F. et al. 1998. Diabetes, vol. 47:764-769; Ahrén, B. et al. 2002. Diabetes Care, vol. 25:869-875; Näslund, E. et al. 1998. Am. J. Clin. Nutr., vol. 68:525-530; Meneilly, G. S. et al. 2003. Diabetes Care, vol. 26:2835-2841.

However, GLP-1 is rapidly degraded in plasma and therefore has a very short half-life of about 1-2 min. The enzyme mainly responsible for degradation of GLP-1 is DPP-IV. Inhibition of DPP-IV might therefore result in prolongation of the circulating half-life of GLP-1, such that GLP-1 levels increase as to be able to act as a therapeutic agent.

DPP-IV has also been shown to be involved in T cell activation and growth. In the immune system, DPP-IV is expressed primarily on the surface of T cells. It has been shown that the expression of DPP-IV is rapidly increasing upon mitogenic or antigenic stimulation. Moreover, it has been shown that inhibition of DPP-IV can suppress the activation of antigen-induced T cell clones and could thus be useful for therapeutic interventions in immune diseases, in particular in autoimmune diseases, such as e.g. MS, and rheumatoid arthritis. Conversely, DPP-IV inhibitors stimulate the production of the immunoregulatory cytokine TGF-β1. See e.g. Reinhold, D. et al. 2000. Cellular Peptidases in Immune Functions and Diseases 2, Langner and Ansorge ed., Kluwer Academic/Plenum Publishers, 155-160; Steinbrecher, A. et al. 2000. Cellular Peptidases in Immune Functions and Diseases 2, Langner and Ansorge ed., Kluwer Academic/Plenum Publishers, 145-153; and Tanaka S. et al., 1997. Int. J. Immunopharmac., vol. 19:15-24.

Currently, several chemical compounds are used in vitro and in animal models to inhibit DPP-IV activity, such as e.g. valine-pyrrolidide (Deacon, C. F. et al., supra), 1-[[[2-[(5-cyanopyridin-2-yl)amino]ethyl]amino]acetyl]-2-cyano-(S)-pyrrolidine (Ahrén, B. et al., supra), Lys[Z (NO₂)]-thiazolidide and Lys[Z(NO₂)]-pyrrolidide (Reinhold, D. et al., supra). However, such chemical compounds have the disadvantage that they often have to be administered by way of injections, and they may result in side effects as chemical drugs often do.

The present inventors have now found that protein hydrolysates, in particular milk protein hydrolysates, more in particular casein protein hydrolysates, are capable of inhibiting DPP-IV, and thus are capable of preventing and/or treating DPP-IV mediated conditions. In contrast therewith, intact proteins are found not to inhibit DPP-IV. The protein hydrolysates according to the present invention have the advantage that they can be administered by means of self medication, they comprise natural compounds, i.e. natural peptides, and are generally regarded as safe, side effects not being known, such that they are generally allowable.

Thus, in a first aspect the present invention relates to the use of one or more protein hydrolysates for inhibiting DPP-IV. Said inhibition of DPP-IV can be employed as to prevent and/or treat deleterious DPP-IV mediated conditions.

Therefore, in a second aspect the present invention relates to the use of one or more protein hydrolysates for the manufacture of a medicament, food supplement, beverage or food product for prophylaxis and/or treatment of a DPP-IV mediated condition.

As herein used, “one or more protein hydrolysates” refers to a mixture of peptides derived from hydrolysis of one or more proteins with a minimal degree of hydrolysis, i.e. the percentage of hydrolysed peptide bonds of the total amount of peptide bonds, of 5%, preferably of 10%, more preferably of 20%, most preferably of 30%. Said protein can be derived from one protein source or may be derived from more protein sources. Examples of such protein sources are microorganisms (yeast, bacteria, fungi), plants (e.g. soy, pea, cotton, corn, wheat), animals, and from animal derived protein sources such as milk, blood, meat, egg and gelatin. Thus, the one or more proteins may be e.g. protein from microorganisms, vegetable protein, animal protein, such as protein derived from meat scraps, fish, crustaceans or mollusks, milk protein and egg protein.

Said hydrolysis of one or more proteins can be performed by any means known in the art. Examples thereof include methods for chemical hydrolysis or enzymatic hydrolysis. Non-limiting examples of methods for chemical hydrolysis are well known in the art and comprise e.g. hydrolysis using cyanogen bromide, acid hydrolysis, e.g. using hydrochloric acid or hydrolysis by means of fermentation of the one or more protein sources comprising the one or more proteins. Non-limiting examples of methods for enzymatic hydrolysis are also well known in the art and comprise hydrolysis using purified enzyme preparations or crude enzyme preparations. Enzyme preparations to be used may comprise endo- or exopeptidases, proteases, or mixtures thereof, and examples thereof include trypsin, chymotrypsins A, B and C, pepsin, rennin, microbial alkaline proteases, papain, ficin, bromelain, cathepsin B, collagenase, microbial neutral proteases, carboxypeptidases A, B and C, carnosinase, anserinase, and many more which are well known to a person skilled in the art. Also combinations of these proteases may be used. Also commercially available enzyme preparations such as e.g. Alkalase, Chymotrypsine 800s, Neutrase (all available from Novo Nordisk, Denmark), Protex 6.0L, Peptidase FP (both available from Genencor, USA), Corolase L10 (Rohm, Germany), Pepsin (Merck, Germany), papain, pancreatin, proleather N and Protease N (Amano, Japan), or combinations thereof may be used. Enzymes prepared by means of recombinant DNA technology may also be used.

The term “prophylaxis” as herein used refers to preventing the emergence of a DPP-IV mediated condition in case no symptoms are as yet observed. As such, the one or more protein hydrolysates may be employed to prevent deleterious DPP-IV mediated conditions from occurring, and may therefore be used to improve or stabilise health of any subject.

As herein used, “a DPP-IV mediated condition” refers to any deleterious condition that arises (at least partially) or deteriorates due to the action of DPP-IV, such that DPP-IV plays an important role in the pathogenesis. Non-limiting examples of a DPP-IV mediated condition are obesity, type 2 diabetes mellitus, and immunological disorders, such as autoimmune diseases, e.g. multiple sclerosis, rheumatoid arthritis, and Graves' disease. Other autoimmune diseases envisioned to benefit from inhibition of DPP-IV are type 1 diabetes mellitus, autoimmune haemolytic anaemia, Hashimoto's thyroiditis, myasthenia gravis, Goodpasture's syndrome, systemic lupus erythematosus, primary biliary cirrhosis, Sjögren's syndrome, chronic active hepatitis, mixed connective tissue disease, scleroderma, and chronic idiopathic thrombocytopenic purpura.

In one embodiment, the one or more protein hydrolysates are used for the manufacture of a medicament, food supplement, beverage or food product for increasing satiety in a subject. As GLP-1 slows gastric emptying and inhibits food intake, a longer circulation half-life of GLP-1 as a result of inhibition of the degradation enzyme DPP-IV will increase satiety in a subject, such that said subject will feel less hungry and have a reduced food intake. In particularly subjects being overweight, such as e.g. obese subjects or subjects being only slightly overweight, will benefit from inhibition of DPP-IV by administration of the one or more protein hydrolysates according to the present invention. The medicament, food supplement, beverage or food product can however also be employed as to retain a certain weight as to not get overweight, and may therefore be used to stabilise and/or improve the body weight as for cosmetic purposes, i.e. for stabilising and/or improving appearance.

Therefore, in a further embodiment the one or more protein hydrolysates of the present invention are used for the manufacture of a medicament, food supplement, beverage or food product for prophylaxis and/or treatment of obesity. For reasons as set out above, administration of the one or more protein hydrolysates according to the present invention is likely to be advantageous in the prophylaxis and/or treatment of obesity.

In another embodiment the one or more protein hydrolysates of the present invention are used for the manufacture of a medicament, food supplement, beverage or food product for lowering of blood glucose levels. It has been found that blood glucose levels are reduced by ingestion of the hydrolysates, resulting in improved glucose management, which is particularly advantageous in diabetic subjects.

In yet a further embodiment, the one or more protein hydrolysates according to the present invention are used for the manufacture of a medicament, food supplement, beverage or food product for prophylaxis and/or treatment of type 2 diabetes mellitus. Type 2 diabetes mellitus is characterised by resistance to insulin, such that the body does not respond to insulin appropriately, resulting in hyperglycaemia. It is often accompanied by obesity. As GLP-1 contributes to normalisation of blood glucose levels as well as to the control of satiety and obesity (body weight), increase of GLP-1 levels by increasing the circulation half-life thereof as a result of inhibition of DPP-IV, it is expected that inhibition of DPP-IV by administration of the one or more protein hydrolysates according to the present invention contributes to prophylaxis and/or treatment of type 2 diabetes mellitus.

In yet another embodiment, the one or more protein hydrolysates according to the present invention are used for the manufacture of a medicament, food supplement, beverage or food product for prophylaxis and/or treatment of an immunological disorder. As discussed above, DPP-IV is thought to play an important role in the pathogenesis of certain immunological disorders. Inhibition of DPP-IV is considered to have a beneficial effect on such immunological disorders, such that the administration of the one or more protein hydrolysates according to the present invention, inhibiting DPP-IV activity, may result in the prophylaxis and/or treatment of such immunological disorder.

It is preferred that such immunological order is an autoimmune disease, as suppression of such diseases by inhibition of DPP-IV has been well established (supra).

Preferably, said autoimmune disease is chosen from the group, consisting of rheumatoid arthritis, multiple sclerosis and Graves' disease, for the same reason as set forth above.

For use in a medicament or food supplement, said preparation can be combined with any suitable carrier, diluent, adjuvant, excipient, etc. in order to obtain the medicament in the desired administration form. Advantageously, said medicament or food supplement is administered orally. The term “food supplement” is known in the art and includes food supplements in the form of a powder or medicament, as well as health products, such as health drinks. An ingredient that can be added to food before consumption or a preparation that can be consumed as such is also encompassed.

For the intended use, the one or more protein hydrolysates according to the invention may be administered alone or in admixture with a pharmaceutically acceptable carrier, in suitable pharmaceutical formulations which are a further object of the invention.

Examples of said formulations, which may be prepared using well known methods and excipients, such as those described in “Remington's Pharmaceutical Sciences Handbook”, Mack Pub. Co., N.Y. U.S.A., are tablets, capsules, syrups, and the like for oral administration, whereas for the parental administration suitable forms are sterile solutions or suspensions in acceptable liquids, implants, etc.

The posology will depend on several factors such as type and seriousness of the pathological conditions to be treated, patient's weight and sex, etc. and will be easily determined by the skilled practitioner.

For use in a beverage or food product, said preparation can be combined with any common food ingredient. The term “beverage” is meant to include cordials and syrups, as well as formulations of a dry powder to be dissolved in water or another liquid component for the preparation of instant drinks.

In another embodiment, the one or more protein hydrolysates are milk protein hydrolysates. It has been found that of all protein hydrolysates tested, milk protein hydrolysates provide the largest inhibition of DPP-IV and are therefore likely to provide the largest effect on the above-mentioned disorders and diseases.

Preferably, said milk protein hydrolysates are casein protein hydrolysates. It has been found that of all milk protein hydrolysates tested, casein protein hydrolysates inhibit DPP-IV the most and are therefore likely to provide the largest effect on the above-mentioned disorders and diseases.

The protein hydrolysates, preferably milk protein hydrolysates, more preferably casein protein hydrolysates, may be fractionated by means of extraction, precipitation, filtration, ultrafiltration, nanofiltration, microfiltration or conventional column chromatography (preferably ion exchange or affinity chromatography), or any combination of the above techniques, as to further isolate the DPP-IV inhibitory activity. As such, a fraction comprising a mixture of peptides or even single peptides may be identified that have an increased inhibitory effect on DPP-IV compared with the protein hydrolysates according to the present invention. Such mixture of peptides or single peptides are also encompassed in the present invention. It is further envisioned that such peptides may be prepared by means of recombinant DNA technology, such as expression of the DNA encoding therefore in a suitable host, or by chemical synthesis.

The molecular weight of such peptides may vary depending on the molecular weight of the one or more protein sources. In case of a high degree of hydrolysis, the peptides will generally be of smaller molecular weight than in case of a lower degree of hydrolysis.

It is preferred that the one or more protein hydrolysates are obtained by enzymatic hydrolysis as discussed above, as enzymatic hydrolysis provides a suitable degree of hydrolysis and is conveniently performed. Moreover, the enzymes employed for enzymatic hydrolysis can be easily separated from the one or more protein hydrolysates by means of simple column chromatography, such as e.g. gelfiltration chromatography, or be inactivated by means of heat, acid, base, or the addition of inhibitors.

Preferably, said one or more protein hydrolysates are administered in an amount of 0.0001-0.1 g/kg body weight, depending type and seriousness of the pathological condition to be treated, weight and sex of the subject, etc. Such factors will easily be determined and taken into account by the skilled practitioner. Using such range, sufficient inhibition of DPP-IV will be achieved as to effect the desired inhibition of DPP-IV required for prophylaxis and/or treatment of the herein disclosed disorders and diseases.

The present invention is also directed to a method for prophylaxis and/or treatment of any DPP-IV mediated condition as discussed above, said method comprising administering an effective amount of one or more protein hydrolysates as discussed above, to a subject in need thereof.

The following examples are employed to further illustrate the present invention, but are in no way meant to limit the scope thereof.

### EXAMPLE 1 IN VITRO MEASUREMENT OF DPP-IV ACTIVITY

DPP-IV activity can be determined by measuring the increase in absorption at 385 nm using Gly-Pro-p-nitroanilide (Sigma G-0513) as DPP-IV substrate. A decrease in DPP-IV activity is a measure for the inhibition.

13.152 mg Gly-Pro-p-nitroanilide (substrate; Sigma G-0513) was dissolved in 1 ml Tris-buffer, pH 8.0. DPP-IV (Sigma D-7052) was diluted with Tris-buffer, pH 8.0 to 1.1 Unit/ml. The substrate was diluted 50-fold with Tris-buffer, pH 8.0. The samples were prepared by diluting a protein hydrolysate to a 1 wt. % protein solution in Tris-buffer, pH 8.0. The samples were then serially diluted to obtain a range of sample concentrations. 50 μl of the different serially diluted samples and 50 μl of the diluted substrate were then pipetted in wells of a microtiterplate with 96 wells. Subsequently, 100 μl of the diluted enzyme was pipetted in each well of a plate with 96 wells. Then the increase in absorption at 385 nm was determined and DPP-IV activities at different concentrations of protein hydrolysates were determined, from which the IC₅₀ (i.e. the concentration of inhibitor (protein hydrolysate) that inhibits 50% of the DPP-IV activity) could be derived. The results are set forth in the table below (All hydrolysates except CE90F and CE90FW having a degree of hydrolysis (DH) of more than 5%). As a control, two unhydrolysed proteins (Sodium caseinate from DMV International, The Netherlands, and Bipro from Davisco Foods, USA) with a degree of hydrolysis (DH) of 0% were tested. The degree of hydrolysis was determined using the o-phtaldialdehyde method, which is well known in the art.

| | IC50 | Stdev | DH | |
|---|---|---|---|---|
| | (ug/ml) | (ug/ml) | (%) | Source |
| Hydrolysate^{a} | | | | |
| CE90STL | 291.51 | 22.50 | 39 | casein |
| CE90MJ | 332.16 | 21.86 | | casein |
| LE80BT | 346.39 | 2.68 | 40 | whey |
| CE90ACE | 347.98 | 61.16 | 13 | casein |
| CE90MK | 363.99 | 41.17 | | casein |
| CE90BTF | 371.18 | 20.99 | | casein |
| AK2-29 | 407.66 | 22.20 | | corn |
| CE90CCP | 471.31 | 24.02 | | casein |
| CE90MS | 497.01 | 16.92 | | casein |
| Peptigen Di-4059 | 502.84 | 15.00 | | pea |
| CE90RC | 509.67 | 7.59 | | casein |
| LE80MX | 511.73 | 16.54 | | whey |
| WE80BH | 515.37 | 12.71 | 17 | whey |
| WE80BH | 517.97 | 24.69 | | whey |
| CE90CPP | 536.42 | 35.62 | 20 | casein |
| D4 | 543.30 | 29.14 | | haemoglobulin |
| WE80M | 550.80 | 13.66 | 15 | whey |
| Cystein Peptide | 578.15 | 32.58 | 28 | whey |
| CE80PS | 620.11 | 33.09 | | casein |
| CE90HM | 624.71 | 75.84 | | casein |
| CE90M | 630.57 | 72.52 | | casein |
| LE80F | 645.34 | 39.16 | 20 | whey |
| CE90MPC | 712.01 | 18.25 | | casein |
| CGE60M | 722.09 | 58.08 | | corn |
| WE80BG | 723.13 | 110.69 | 29 | whey |
| CE90GMM | 725.92 | 8.49 | 27 | casein |
| EE90FX | 736.36 | 41.38 | 12 | casein + egg |
| CE90B | 739.03 | 73.77 | 17 | casein |
| CE90GF-US | 740.70 | 73.29 | | casein |
| LE80PS | 746.53 | 45.48 | 67 | whey |
| CE90BT | 768.69 | 57.02 | | casein |
| LE80GF | 777.56 | 38.26 | 15 | whey |
| GK 01-21 | 779.20 | 26.89 | | corn |
| LE80GF-US | 797.18 | 97.94 | | whey |
| CNE50M | 803.46 | 28.24 | | cotton |
| LE80F-EU | 813.60 | 73.48 | | whey |
| CE90GBT | 814.67 | 51.68 | 19 | casein |
| PCE80BT | 839.95 | 52.17 | | pea |
| LE80BM | 847.51 | 25.50 | | whey |
| LE80BH | 847.51 | 20.82 | | whey |
| CR90ML | 867.60 | 16.41 | | casein |
| LE80GF-EU | 884.56 | 59.45 | | whey |
| AED80M | 923.19 | 19.56 | | meat |
| SE70BT | 979.90 | 81.86 | 26 | soy |
| AE80M | 1051.80 | 103.97 | | meat |
| SE50MAF-UF | 1066.20 | 32.82 | | soy |
| GK01-31B | 1089.50 | 84.33 | | wheat |
| WGE80GPA | 1104.71 | 90.32 | | wheat |
| CE90MX | 1128.95 | 39.78 | | casein |
| WGE80GP | 1142.67 | 269.42 | | wheat |
| SE50MAF | 1164.33 | 11.48 | | soy |
| EH 07-92 A | 1165.27 | 15.80 | | pea |
| SE50MK | 1188.48 | 158.96 | | soy |
| SE50BT | 1238.90 | 41.11 | 28 | soy |
| GS90FB | 1248.81 | 111.06 | | soy |
| WGE80M | 1281.97 | 266.07 | | wheat |
| WGE80GPN | 1381.47 | 139.03 | | wheat |
| WGE80BT | 1747.60 | 48.95 | | wheat |
| WE80FG | 1757.94 | 156.76 | 7 | whey |
| WGE80F | 1888.67 | 204.55 | | wheat |
| WGE80GPU | 2702.83 | 528.82 | | wheat |
| PP90M | 2785.60 | 77.13 | | gelatin + |
| | | | | yeast |
| GE50MB | 2908.31 | 738.10 | | gelatin |
| GE90MB | 2922.73 | 285.05 | | gelatin |
| GE90M | 2932.98 | 452.98 | | gelatin |
| BV50 | 3433.32 | 194.02 | | casein |
| CE85W | 3644.82 | 309.55 | | casein |
| WE90F | 4064.77 | 285.69 | 6 | whey |
| ME50MB | 6598.27 | 740.14 | | malt |
| CE90F | N.D. | — | 4 | casein |
| CE90FW | N.D. | — | 4 | casein |
| Unhydrolysed | | | | |
| protein | | | | |
| Sodium Caseinate | N.D. | — | 0 | casein |
| Bipro | N.D. | — | 0 | whey |

N.D. = not detectable
^{a}All protein hydrolysate preparations are commercially available from DMV International, The Netherlands (with the exception of Peptigen Di-4059, which is available from ARLA Foods, Denmark), or in-house preparations.

### EXAMPLE 2 GLUCOSE TOLERANCE TEST

A total of 10 subjects diagnosed with type 2 diabetes mellitus was recruited for the experiment. Exclusion criteria were the following: fasting glucose concentrations lower than 7.2 mmol/l or higher than 10.0 mmol/l, mean Hb1c levels lower than 6.3% or higher than 10% and a BMI lower than 19 kg/m² or higher than 29 kg/m². Also excluded were subjects that were pregnant or breast feeding or with evidence of diabetic organ diseases or any other relevant clinical conditions. Each subject completed four oral glucose tolerance tests, two using a placebo and two using CE90STL (DMV International, The Netherlands). The placebo consisted of a mix of free amino acids having the same amino acid composition as CE90STL. After an overnight fast the subjects orally consumed 75 grams of glucose with either 1 g placebo or 1 g CE90STL. Arterialised blood was obtained using a catheter inserted into the dorsal hand vein. Blood samples were obtained immediately before ingestion of the glucose load and for 3 hours every 30 minutes thereafter. Blood samples were analysed for glucose using the glucose oxidase method from Boehringer Mannheim (Germany), and for insulin using a radioimmunoassay (Pharmacia, Uppsala, Sweden).

Results are expressed as area under the curve (AUC) and analysed by a two-way analysis of variance (ANOVA). Differences were considered significant using a p-value of 0.05. The results are shown in the table below.

| | Glucose | Insulin |
|---|---|---|
| | (mmol/l · h) | (pmol/l · h) |
| Placebo | 5.1 (0.5) | 451 (63) |
| CE90STL | 3.9 (0.7) | 432 (54) |

The results show a significant lowering of the postprandial glucose levels but not of the insulin levels. The absence of a significant effect on the insulin levels indicates that DPP-IV inhibition improves the overall insulin response which results in improved glucose management in these diabetic subjects.

1-13. (canceled)

14. A method for the prophylaxis and/or treatment of a DPP-IV mediated condition, comprising administering to a subject in need thereof, an effective amount of one or more protein hydrolysates.

15. The method according to claim 14, for increasing satiety in a subject.

16. The method according to claim 14, for prophylaxis and/or treatment of obesity.

17. The method according to claim 14, for lowering of blood glucose levels.

18. The method according to claim 14, for prophylaxis and/or treatment of type 2 diabetes mellitus.

19. The method according to claim 14, for prophylaxis and/or treatment of an immunological disorder.

20. The method according to claim 19, for prophylaxis and/or treatment of an autoimmune disease.

21. The method according to claim 20, wherein the autoimmune disease is chosen from the group consisting of rheumatoid arthritis, multiple sclerosis and Graves' disease.

22. The method according to claim 14, wherein the one or more protein hydrolysates comprise milk protein hydrolysates.

23. The method according to claim 22, wherein the milk protein hydrolysates comprise casein protein hydrolysates.

24. The method according to claim 14, wherein the one or more protein hydrolysates are obtained by enzymatic hydrolysis.

25. The method according to claim 14, wherein said one or more protein hydrolysates are administered in an amount of 0.0001-0.1 g/kg body weight.

26. The method according to claim 14, for inhibiting DPP-IV.

27. The method according to claim 14, wherein said one or more protein hydrolysates are incorporated in a medicament, food supplement, beverage or food product.
